(19) United States
(12) Patent Application Publication
(10) Pub. No.: US 2023/0200873 A1
(43) Pub. Date: Jun. 29, 2023

(54) Dental and Orthopedic Insertion Tool

(71) Applicant: M3 HEALTH INDÚSTRAIA E COMÉRCIO DE PRODUTOS MÉDICOS, ODONTOLÓGICOS E CORRELATOS S.A., Jundiai (BR)

(72) Inventors: Alberto BLAY, São Paulo (BR); Alex Araújo NUCINI, Santa Rosa de Viterbo (BR); Jamil Awad SHIBLI, Guarulhos (BR); Samy TUNCHEL, São Paulo (BR)

(21) Appl. No.: 17/928,122

(22) PCT Filed: May 28, 2021
(86) PCT No.: PCT/BR2021/050228

§ 371 Date: Nov. 28, 2022

(30) Foreign Application Priority Data
May 28, 2020 (BR) 102020010700-3

(51) Int. Cl. A61B 17/88; A61C 8/00

(52) U.S. Cl. CPC A61B 17/88; A61C 8/0089; A61B 2090/031

(57) ABSTRACT

The present invention describes a dental insertion tool, comprising: an axle and an insertion element; wherein the axle comprises a axle attaching region; the insertion element comprises a attaching region of the insertion element; in which axle's attaching region and the insertion element's attaching region make the connection between the said axle and the said insertion element; the axle rotating the insertion element when connected; wherein the axle fixation region comprises a torque limiting element, which may be an shear pin and a hole located in the attaching region of the insertion element or a latch and spring lock set wherein the latch hooks in the internal grooves of the insertion element's attaching region.

Thus, one advantage of the present invention is the torque limiting element is configured in order to limit the torque applied to the implant, and then avoiding the possible damage that can be caused to the implant and the patient, preserving the implant even if a excessive torque is applied, depending on the type of application, implant and/or technique used.

### TECHNICAL FIELD

The present invention relates to the field of tools, more precisely applied to tools for dental implants and orthopedic prostheses comprising a shear pin.

Further, the present invention relates to the field of tools, more precisely applied to tools for dental implants and orthopedic prostheses comprising a latch and spring system.

### BACKGROUND ART

Ratchet-type keys are essential tools for ergonomics and agility and can be used in several areas. With them, injuries can be avoided by both effort and impact, because these tools allow the user to tighten a screw or nut repeatedly without always repositioning the tool, since the inner ratchet keeps the socket in place while the key arm is repositioned.

There are ratchet key models that may or may not be reversible and are marketed in the most commonly sizes in the socket fittings on the market (1/4, 3/8, 1/2, 3/4, among others). In addition to the fittings, there are the tips of the sockets that will come into contact with the nuts and screws that, in turn, are also available in wide variety in view of the diversity of screws and nuts with different types of fittings and sizes (hexagon, straited, torx, philips, alien, among others).

Currently the market offers a great diversity of accessories and accessories for the most diverse applications, being that these are usually sold in kits along with the ratchet keys. Much of these is produced in a special steel alloy that receives chromium and vanadium in its composition to increase durability and work resistance.

Additionally, it is common to use silicone o'rings with the ratchet wrench to promote the mechanical retention of a second wrench. In the field of dentistry, for example, this measure avoids direct contact of the user/professional with the key/instrument that will contact the patient's body during the surgical procedure. However, it should be considered that instruments of this nature need to be sterilized before each use. Thus, in the case of silicone rings, they are expected to gradually lose their properties due to the effect of high temperature during autoclaving process, so that they no longer firmly retain the insertion key. On the other hand, metal rings also lose their spring effect properties in view of natural wear related to time of use.

In this sense, patent document MU 8601138-3 describes a support instrument for oral-maxilla-facial surgery that allows the surgeon at the time of surgery to manipulate the prosthesis pin more easily and maintain total control over the forces applied so that the pin penetrates properly and safely into the bone structure of the jaw. It is a tool of surgical aid that effectively performs the described technique making it possible to perform all steps of pin screw or osseointegration element in the patient's jaw, as well as its own torqueing in the definitive location, with the innovation of the tool be designed to stabilize naturally by the existence of specifically designed fittings and anatomical handles, inferring better grip for the surgeon, improving the effectiveness and surgical performance.

Patent document MU 8700367-8 describes an improvement in the clinical procedure in the manufacture of implant screws. The invention is characterized by a single main body formed by a cylindrical head below which there is a screw whose area is equally cylindrical, while the lower end of said main body projects a conical tip that ends up on a flat base, and the said head is provided internally in its upper section of chamfer followed immediately by a hexagon endowed at its vertices of coupled channels for insertion of torque tool , while its base projects a hole equipped with thread for fixation of the implant, being the said conical tip externally and provided, longitudinally, with conductive grooves.

Patent document MU8600685-1 describes a torque tool for implant by forced penetration, pins or elements of dental osseointegration, endowed with male thread in its penetration part that will make the function of supporting the false tooth in the jaw and, called; key for fixation of dental prosthetic element of osseointegration, project developed on a slender tarugo of metallic structure containing at its upper end a tip with hexagonal torque pin, which is coupled in the internal hexagonal fitting cavity of the osseointegration element, allowing the task of taper.

Patent document BR 102017018084-0 describes a dental tool that allows the function of capture and fixation without the need for tool change, with lower possibilities of occurring flaws in the component/screw at the time of torque application (screw/component head tightening) and, which can still be used in diameter components smaller than 1.4 mm.

Document BR112019003751-9 refers to a dental implant wrench, having an axle part manufactured in an intelligent alloy type that bends with little or no resistance along a respective arc formation part and without transmitting torque forces along a portion of the respective distal front end on which is connected a guide tip designed to interact with an appropriate-sized screw. The smart alloy automatically takes its original form in the absence of torsion forces on the axle along the arc forming part. The dental implant key has a very narrow design profile and is sized to attach to an elastic pin puller or activator for dental implant designs with Cone Morse prosthetic connection. This allows the removal of a pin immediately after removal of the screw or, alternatively, the tightening of a pin without the use of a screw.

Patent document PI 0902114-0 describes a device that transforms angular linear motion into a rotating radial motion for screwing the intermediate part to the dental implant base with the surgeon's use of only one hand. To this end, the device in question was developed from a cylindrical handle body by housed elements arranged in the form of a compression set and a torque set. After adjusting the device, according to the torque required for screwing, the compression assembly locks the torque assembly and the surgeon, wielding the cylindrical body with one hand, directs and attaches the tightening wrench directly to the intermediate part, then always performing with only one of the hands the radial rotation from the cylindrical body.

Patent document MU 8401655-8 describes a dental implant with internal torque that has a composite connection to an internal hemise, a connection for use in contra angle and a connection for use in ratchet key torquemeters, and, other aspects, causing the elimination of the insertion system aided by assembler, eliminating possible deformations to the external hexagon of the implant and consequent undesirable adaptations of the prosthetic component abutment.

A recurrent problem in the use of such tools is the excess torque applied by the user/professional to the implant at the time of insertion thereof in the bone. If the torque is greater than a certain limit, the prosthetic adaptation can be compromised and cause problems for the patient such as bone microfractures adjacent to the implant, necrosis from heating during implant torque, and/or bone tablet fractures of atrophic structures.

In this sense, this patent application presents a solution that has technical differentials in order to limit the force applied to implants and thus avoid the possibility of harm to patients, as will be disclosed below.

### OBJECTIVES OF INVENTION

Thus, it is an objective of the present invention to provide a solution to the challenges and limitations listed above, presenting a dental insertion tool with an analog torque limitation, in order to limit the torque applied and protect the implant and the patient.

It is an objective of the present invention to provide two options of torque interruption, offering versatility and power of choice to the medic professional.

It is also an objective of the present invention to provide a safe, replicable, and reliable lock, with the breakpoints being always the same.

It is also an objective of the present invention to provide a tool for the insertion of orthopedic prostheses with the same advantages as the dental tool.

It is also an objective of the present invention to provide a lock of simple and practical use for the professional medic without causing an increase in the cost of the parts.

### SUMMARY OF INVENTION

The present invention achieves this and other objectives by providing a dental and orthopedic insertion tool, comprising an axle; and an insertion element; wherein the axle comprises a axle attaching region; the insertion element comprises a attaching region of the insertion element; wherein the axle attaching region and the insertion element attaching region provides the connection between the said axle and the said insertion element; the axle rotating the insertion element when connected; where the axle attaching region comprises a hole and the insertion element attaching region comprises a hole; the tool also comprises a force-limiting element, which interrupts torque transmission when applying a torque.

According to a first preferred embodiment, the force-limiting element is a shear pin configured to fix the insertion element on the axle through the holes. The pin is sized to withstand a maximum rotation force, wherein the shear pin is broken when the torque is greater than the torque supported by the shear pin. The maximum torque supported by the shear pin is related to the diameter of the shear pin, in which the shear pin is configured to withstand a maximum rotation force between 20 N·cm to 575 N·cm, with a diameter of the shear pin ranging from 0.5 mm to 2.0 mm. The insertion element is preferably configured to connect to a dental implant.

According to a second preferred embodiment, the force-limiting element is a system comprising an internal latch and spring, configured so that the latch is pressed by the spring against the internal grooves to transmit the torque applied on the axle to insertion element and the latch is driven away by the spring when the maximum torque is exceeded, interrupting the torque transmission.

The dental and/or orthopedic insertion tool can preferably be manufactured in Stainless Steel, with a length ranging from 16 mm to 40 mm, and the tool contains adaptation of the type contra angle and/or ratchet.

### DETAILED DESCRIPTION OF DRAWINGS

The present invention will be more detailed and elucidated based on the following figures:

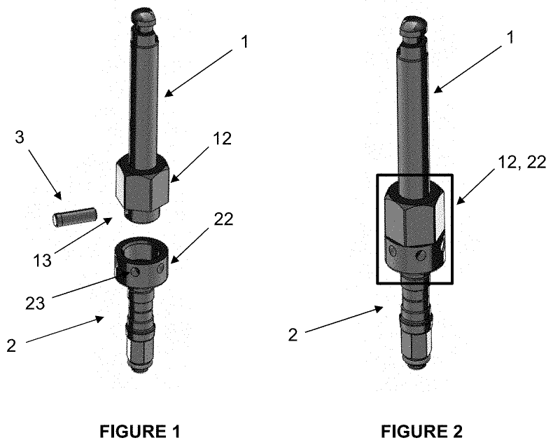

FIG. 1 illustrates a dental insertion tool according to a first configuration of the present invention, illustrating the elements that compose it;

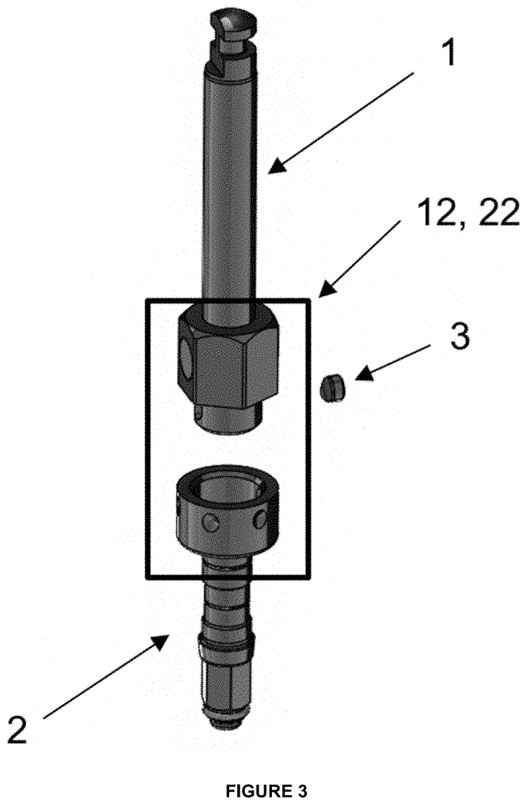

FIG. 2 shows a dental insertion tool according to a first configuration of the present invention, illustrating the elements that compose it put together;

FIG. 3 shows a dental insertion tool according to a first configuration of the present invention, illustrating a shear pin broken by excess force applied to the torque.

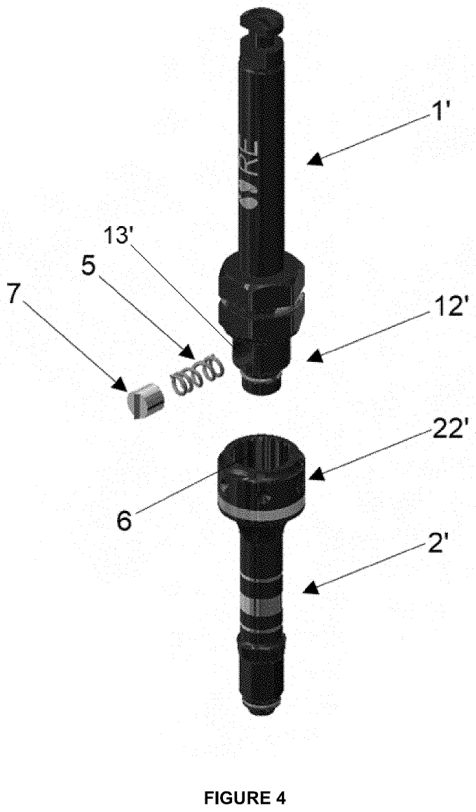

FIG. 4 illustrates a dental insertion tool according to a second configuration of the present invention, illustrating the elements that compose it;

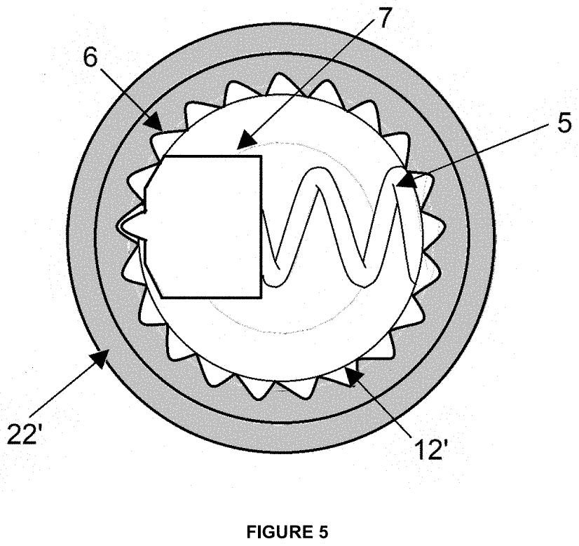

FIG. 5 shows the internal detail of the second configuration, with the spring pressing the latch against the internal groove of the insertion element; and

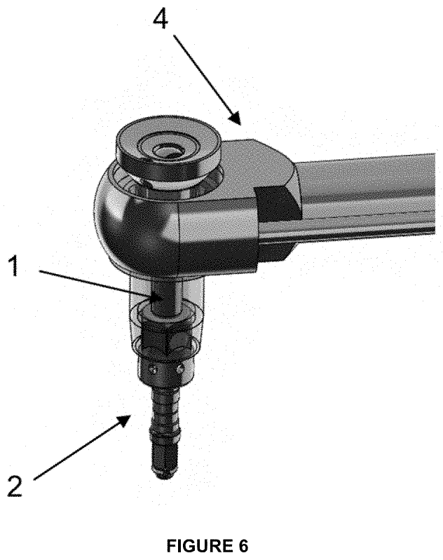

FIG. 6 illustrates a dental insertion tool according to a preferred configuration of the present invention, of ratchet type.

### DETAILED DESCRIPTION OF THE INVENTION

The present invention refers to one the tool of dental and orthopedic insertion comprising an element limiting applied force.

The tool according to the present invention is preferably manufactured in temperate stainless steel.

The connection between these two parts is made by means of the torque-limiting element, fixed to holes located on the axle and the insertion element for fixing them, and can be strategically arranged in order to better fit the key, according to FIG. 6. Preferably the attaching region of the present invention comprises one or more holes to better meet the technique for insertion of dental implants with adaptation of the key type contra angle and/or ratchet.

Regrading the dimensions, the length of the tool can vary between 16 mm and 40 mm, presenting a preferably hexagonal or square shape.

In reference to FIGS. 1 and 2, in a first embodiment, the torque-fixing element is a shear pin, configured to limit the torque applied to the implant, thus avoiding the possible damage that can be caused to the implant and the patient.

Laboratory tests determined that the shear pin with a diameter of 0.90 mm supported 80 N·cm, 0.95 mm supported 92 N·cm and 1 mm supported 105 N·cm. This way, it was determined the equation of F=200×d²−130×d+35 wherein F is the supported force (in N·cm) and d is the diameter (in mm). Thus the shear pin can vary between 0.5 mm and 2 mm, supporting a force (directly related to diameter) between 20 N·cm 575 N·cm.

In view of the above teachings, the present invention describes a dental insertion tool comprising an axle 1 and an insertion element 2; where axis 1 comprises a axle attaching region 12; the insertion element 2 comprises a attaching region of insertion element 22; in which the axle attaching region 12 and the attaching region of the insertion element 22 make the connection between axle 1 and insertion element 2; the axle 1 rotating the insertion element 2 when connected; wherein the axle attaching region 12 comprises a hole 13 and the attaching region of the insertion element 22 also comprises a hole 23; the tool 4 further comprising a shear pin 3, in which the shear pin 3 is configured to fix the insertion element 2 on axle 1 through of holes 13, 23.

In reference to FIG. 4 a second configuration, the force limiting element is a system of an internal latch and spring, configured to limit the torque applied to the implant, thus avoiding the possible damage that can be caused to the implant and the patient.

In view of the above, the present invention describes a dental insertion tool comprising a axle 1′ ;and an insertion element 2′; in which the axle 1′ comprises a axle attaching region 12′; and the insertion element 2′ comprises a attaching region of the insertion element 22′; where in axle attaching region 12′ and the attaching region of the insertion element 22′ make the connection between axle 1′ and insertion element 2′; axle 1′ rotating the insertion element 2′ when connected; where the axle attaching region 12′ comprises a latch 7 and a spring 5 inside a hole 13′ and the attaching region of the insertion element 22′ comprises internal grooves 6. The latch 7 which is pressed by spring 5 against the internal grooves 6 of the insertion element 2′ causing the latch 7 to operate inside the groove.

### Device Operation

The axle 1, 1′ is tightly fitted to tool 4, as shown in FIG. 4. Thus, when the torque is applied by the professional in the said tool 4, the same torque is applied to axis 1, 1′, which in turn transmits the torque for the insertion element 2, 2′, which finally transmits the torque to the implant or prosthesis screw.

Due to the fact the fixing between the axle 1, 1′ and the insertion element 2 is made by the torque fixing element, in the hole 13, 13′, the interruption of the torque by the torque fixing element prevents the torque from being passed to the insertion element 2, 2′.

According to the first preferred embodiment, the shear pin 3 is configured to withstand a maximum rotation force, wherein the shear pin 3 is broken when the rotation force is greater than the maximum force supported by the said shear pin 3. The maximum force supported by shear pin 3 is related to the diameter of the shear pin 3, wherein the shear pin 3 is configured to withstand a maximum rotation force between 20 N·cm to 575 N·cm, with a diameter of the shear pin 3 ranging from 0.5 mm to 2.0 mm. The insertion element is preferably configured to connect to a dental implant.

The advantages of this first configuration are that the shear pin is configured to limit the torque applied to the implant, thus avoiding the possible damage that can be caused to it, depending on the type of application, implant and/or technique used. Thus, if the torque at the time of insertion of the implant is greater than the torque for which the pin was sized, the pin will break, ensuring that excess torque will not be transmitted to the implant, preserving the implant as illustrated in FIG. 3.

Another advantage is the replicability and reliability of the shear pin 3. With a standard part in the described sizes, it is possible to offer pins that always shear at a certain torque threshold, being a safe, failproof tool.

Additionally, because it is an analog and physical system, torque interruption always happens, and is not subject to electronic failures that may occur.

According to the second embodiment, the latch 7 is pressed by spring 5 against the internal grooves 6 in a way that it makes the transmission of the torque applied on axle 1 to insertion element 2′. When the torque reaches the value determined by spring pressure 5 is deformed and lock 7 moves away from slots 6, causing shaft 1′ to stop transmitting torque to insertion element 2′, thus avoiding possible damage that can be caused to the same and the patient, preserving the implant.

The torque is defined by the pressure that spring 5 does on latch 7 against the internal grooves 6 of the fixing region of the insertion element 22′, depending on the desired torque it will be necessary to change the dimensions of the spring 5. Thus, the maximum torque responsible for contracting spring 5 and disfitting the latch 7 is directly related to the dimensions of spring 5, and the spring is preferably made of a wire with a diameter between 0.15 mm and 0.5 mm, resulting in compression forces between 0.15 and 10 Kgf, respectively.

Furthermore, the latch is preferably 0.5 to 3.0 mm in diameter, and the arrangement of the internal grooves 6 is such that the angulation of the grooves can be from 30 to 150° and the inclinations between the sides may not be symmetrical, according to the profile illustrated in FIG. 5.

Additionally, other elements with elastic strength can replace spring 5 in alternative embodiments of the present invention without causing damage to the operation of the tool.

One of the advantages of this second configuration is that the latch 7 moves with the torque applied and goes back to its original state when the torque is below the specified, without shearing, which makes this part reusable.

Further, because it is an analog and physical system, torque interruption always happens, and is not subject to electronic failures that may occur.

Additionally, the dental insertion tool 4 can be manufactured in stainless steel, with a length ranging from 16 mm to 40 mm, and tool 4 being angled and/or ratcheted.

Also, tool 4 can have its size suited to be used in orthopedic cases, as a tool for inserting prosthetic screws with a length ranging from 150 mm to 250 mm., and tool 4 being angled and/or ratcheted.

Having described examples of preferred embodiments of the present invention, it should be understood that the scope of the present invention covers other possible variations of the inventive concept described, being limited only by the content of the claims, including the possible equivalents.

1. Dental and orthopedic insertion tool (4), comprising:
an axle (1); and
an insertion element (2);
wherein the axle (1) comprises an axle attaching region (12);
the insertion element (2) comprises an insertion element attaching region (22);
wherein the axle attaching region (12) and the insertion element attaching region (22) connects the axle (1) and the insertion element (2);
the axle (1) capable of rotating the insertion element (2) when connected; the axle attaching region (12) comprising a hole (13) and the insertion element attaching region (22) comprising another hole (23);
the tool (4) further comprising a torque fixing element configured to fix the insertion element (2) on the axle (1) through the holes (13, 23);
characterized by the fact the torque fixing element is a shear pin configured to withstand a maximum rotation torque, so that the shear pin is broken when the rotation torque is greater than the maximum torque supported by the said shear pin, interrupting the torque transmission between the axle and the insertion element.

2. The insertion tool (4) according to claim 1, characterized by the fact the insertion element (2) is configured in order to connect to a dental implant, the tool (4) length ranging between 16 mm and 40 mm.

3. The insertion tool (4) according to claim 1, characterized by the fact the insertion element (2) is configured in order to connect to an orthopedic prosthesis, the tool (4) length ranging between 150 mm and 250 mm.

4. The insertion tool (4) according to claim 1, characterized by the fact it is made of stainless steel.

5. The insertion tool (4) according to claim 1, characterized by being a tool (4) as contra angle and/or ratchet type.

6. The insertion tool (4) according to claim 5, characterized by the fact the maximum torque supported by the said shear pin (3) is related to the diameter of the said shear pin.

7. (canceled)

8. The insertion tool (4) according to claim 6, characterized by the fact the shear pin (3) is configured to withstand a maximum rotation torque between 20 N·cm to 575 N·cm, with a shear pin diameter (3) ranging from 0.5 mm to 2.0 mm.

9-11. (canceled)

12. A dental and orthopedic insertion tool (4), comprising:
an axle (1′); and
an insertion element (2′);
wherein the axle (1′) comprises a. axle attaching region (12′); the insertion element (2′) comprises an insertion element attaching region (22′);
wherein the axle attaching region (12′) and the insertion element attaching region (22′) connect the axle (1′) and the insertion element (2′);
the axle (1′) capable of rotating the insertion element (2′) when connected; the tool (4′) further comprising a torque fixing element configured to fix the insertion element (2′) on the axle (1′) through a hole (13′).
characterized by the fact the by the torque fixing element is a latch (7) connected to a spring (5) and the attaching region of the insertion element (22′) further comprises internal grooves (6), wherein the relaxed spring (5) presses the latch (7) against the internal grooves (6) and when the torque applied by the user reaches the value determined by the spring pressure (5), the said spring (5) is deformed and the latch (7) moves away from the grooves (6), interrupting the torque transmission between the axle (1′) and the insertion element (2′).

13. The insertion tool (4) according to claim 12, characterized by the fact the insertion element (2′) is configured to connect to a dental implant, the tool (4′) length ranging between 16 mm and 40 mm.

14. The insertion tool (4) according to claim 12, characterized by the fact the insertion element (2′) is configured in order to connect to an orthopedic prosthesis, the tool (4′) length ranging between 150 mm and 250 mm.

15. The insertion tool (4) according to claim 12, characterized by the fact it is made of stainless steel.

16. The insertion tool (4) according to claim 12, characterized by being a tool (4′) as contra angle and/or ratchet type.

17. The insertion tool (4) according to claim 16, characterized by the fact the maximum torque responsible for contracting the spring (5) and detaching the latch (7) being related to the dimensions of the spring (5), and the spring is made of a wire that ranges 0.15 mm to 0.5 mm in diameter, resulting in compression forces between 0.15 to 10 Kgf, respectively.

18. The insertion tool (4) according to claim 17, characterized by the fact the spring (5) is configured to be triggered with a maximum rotation torque between 20 N·cm to 575 N·cm, releasing the latch (7).
